(19) United States
(12) Patent Application Publication
(10) Pub. No.: US 2018/0228425 A1
(43) Pub. Date: Aug. 16, 2018

(54) UTERINE ACTIVITY TOOL

(71) Applicant: CLINICAL COMPUTER SYSTEMS, INC., ELGIN, IL (US)

(72) Inventors: Lisa A. MILLER, Portland, OR (US); Randall Iverson PAUL, Mission Viejo, CA (US); Cynthia L. BELL, Woodridge, IL (US); Marianela SPUNG, Wauconda, IL (US); Caren S. BUSEN, Timewell, IL (US); Bradley Joshua BELL, Aurora, IL (US); Ning Xiang LI, Sequim, WA (US); Anne Catherine URMANSKI, Roswell, GA (US)

(73) Assignee: CLINICAL COMPUTER SYSTEMS, INC., ELGIN, IL (US)

(21) Appl. No.: 15/897,905

(22) Filed: Feb. 15, 2018

Related U.S. Application Data
(60) Provisional application No. 62/459,420, filed on Feb. 15, 2017.

(51) Int. Cl. A61B 5/00; A61B 5/0488

(52) U.S. Cl. CPC A61B 5/4356; A61B 5/02411; A61B 5/4325; A61B 5/04882

(57) ABSTRACT

Uterine activity tools and methods of use thereof. The uterine activity tools of this invention are designed to provide clinicians with information to evaluate uterine activity of a pregnant patient utilizing a fetal monitor with a tocodynamometer external pressure sensor (“TOCO”) and/or an intrauterine pressure catheter (“IUPC”). The tool of this invention displays vital information for assessment and documentation of uterine activity including date, time, mode, contraction frequency, contraction duration, contraction intensity, resting tone, relax time, calculated Montevideo units, and number of contractions in a selected time period.

### CROSS REFERENCE TO RELATED APPLICATION

This application claims the benefit of U.S. Provisional Patent Application, Ser. No. 62/459,420, filed on 15 Feb. 2017. The co-pending Provisional Application is hereby incorporated by reference herein in its entirety and is made a part hereof, including but not limited to those portions which specifically appear hereinafter.

### BACKGROUND OF THE INVENTION

### Field of the Invention

This invention relates generally to a system and a method for evaluation and assessment of uterine activity of a pregnant patient utilizing a workstation with a fetal monitor, such as a tocodynamometer external pressure sensor (TOCO) or an intrauterine pressure catheter (IUPC).

### Discussion of Related Art

Recently the obstetrics industry shifted its focus to research of accurate assessment and management of uterine activity. The industry has found that excessive uterine activity was associated with abnormal fetal heart rate (FHR) patterns, presumably because of inadequate uterine relaxation time. Shortened relaxation time can lead to excess constriction of the spiral arterioles that provide oxygen delivery to the placenta and fetus, effectively reducing perfusion pressure and thus placenta blood flow.

In 2008, the National Institute of Child Health and Human Development (NICHD) and the American Congress of Obstetricians and Gynecologists (ACOG) worked on a proposal and update to the classification system and terminology for describing uterine activity patterns. The preferred term to describe excessive uterine activity is now known as tachysystole (TS), which is defined as >5 contractions in 10 minutes, averaged over a 30 minute window. In a recent study, it was also discovered that the use of oxytocin or misoprostol, an epidural, hypertension, and induction of labor were associated with an increased risk of TS and the number of TS events have been associated with an increased risk of cesarean delivery compared to vaginal delivery.

Worldwide, cesarean sections (CS) are becoming a concern because CS have quadrupled in less than 2 decades, making CS the most common surgical procedure performed in women of childbearing age. In the United States, the rate is 32%, one of the highest in the world. These new results suggest that clinicians need to start paying closer attention to uterine activity during labor. It is important for clinicians to reach consensus on definitions related to excessive uterine activity and recognize that the term TS addresses only one aspect of uterine activity, that of frequency. Other parameters such as strength, duration, resting tone, and relaxation time are equally important components of the evaluation of uterine activity during labor. Having this global understanding is crucial to the promotion of improved outcomes for both the mom and the fetus.

As such, there is an ongoing need for an improved system and method for assessment and interpretation of uterine contractions.

### SUMMARY OF THE INVENTION

The present invention provides a uterine activity tool and method to support clinicians during the assessment of uterine activity during labor. The goal of the present invention is to aid clinicians during the decision making process by providing key information to help the clinician evaluate and decide on management of uterine activity, preferably at a patient's bedside. The tool of this invention will serve as an adjunct to individual clinical judgment, allowing the clinician to confirm bedside assessments.

At a high level, the uterine activity tool will be able to allow the clinician to select and analyze a uterine activity tracing segment during the intrapartum period. Preferably, the tool of this invention will provide a clinician with a uterine activity tracing showing and identifying a start point, a peak, an end point and any other important characteristic of each contraction and calculating a number of important characteristics of the contraction to assist the clinician to evaluate the contractions. The uterine activity (UA) tool of this invention also preferably allows the clinician to modify the start point, the peak and/or end point of the contraction (emphasized area) by simply clicking on the strip or tracing, before any calculation takes place. The tool of this invention displays uterine contraction average frequency based on a 10, 15, 30, or 60 min segment at any time period defined by the clinician. In an embodiment of this invention, the tool can display uterine contraction duration when a tocotransducer (TOCO) is being utilized. In another embodiment, the tool may display uterine contraction intensity, duration, relaxation time, and resting tone when an intrauterine pressure catheter (IUPC) is utilized. Use of the IUPC also allows the tool to calculate Montevideo units (MVUs). The tool of this invention may also allow the clinician to add, edit, or remove contractions if results do not match the clinician's visual assessment. The tool allows the clinician to confirm tool evaluation results or clinician's modifications and add UA information to notes and flowsheets. Clinicians are able to utilize the Uterine Contraction Evaluation tool on active traces as well as archived traces. The UA tool of this invention also provides a robust interface capable of sending the information to any EHR system.

Other advantages will be apparent to those skilled in the art from the following detailed description taken in conjunction with the drawings.

### BRIEF DESCRIPTION OF THE DRAWINGS

These and other objects and features of this invention will be better understood from the following detailed description taken in conjunction with the drawings, wherein:

FIG. 1A shows a schematic representation of a uterine activity system according to an embodiment of this invention.

FIG. 1B shows another schematic representation of the uterine activity system according to another embodiment of this invention.

FIG. 1C shows another schematic representation of the uterine activity system according to a third embodiment of this invention.

FIG. 2 shows a screenshot of a trace review screen on a workstation monitor, with a menu providing clinician access to the uterine activity system of this invention.

FIG. 3 shows a screenshot of a uterine activity tool according to an embodiment of this invention.

FIG. 4 shows a screenshot of a portion of the uterine activity tool according to another embodiment of the invention.

FIG. 5 shows the screenshot of the uterine activity tool shown in FIG. 3 showing a drop-down context menu.

FIG. 6 shows a screenshot of the uterine activity tool according to another embodiment of the invention.

FIG. 7 shows another screenshot of the uterine activity tool with a uterine contraction note entry section enabled.

FIG. 8 shows another screenshot of a portion of the uterine activity tool with a mode field indicating Toco mode.

FIG. 9 shows another screenshot of a portion of the uterine activity tool with a mode field indicating IUPC mode.

FIG. 10 shows another screenshot of the uterine activity tool with a UC note entry section enabled.

FIG. 11 shows a screenshot of a notes entry screen according to an embodiment of the invention.

FIG. 12 shows a screenshot of a notes review screen according to an embodiment of the invention.

FIG. 13 shows a screenshot of a notes review screen according to another embodiment of the invention.

FIG. 14 shows a screenshot of a flowsheet screen according to an embodiment of the invention.

FIG. 15 shows a screenshot of a labor quality flowsheet showing uterine contractions according to another embodiment of the invention.

FIG. 16 shows a partial screenshot of an Intensive Maternal assessment flowsheet according to another embodiment of the invention.

FIG. 17 shows a screenshot of a flowsheet according to another embodiment oaf the invention.

FIG. 18 shows a screenshot of a flowsheet according to another embodiment of the invention.

### DEFINITIONS

Within the context of this specification, each term or phrase below will include the following meaning or meanings.

Frequency: Contraction frequency overall generally ranges from 2 to 5 per 10 minutes during labor, with lower frequencies seen in a first stage of labor and higher frequencies during a second stage of labor.

Duration: Contraction duration remains fairly stable throughout the first and second stages, ranging from 45-80 seconds and not generally exceeding 90 seconds.

Strength: Uterine contractions generally range from peaking at 40-70 mmHg in the first stage of labor and may rise to over 80 mmHg in the second stage. Contractions palpated as “mild” would likely peak at less than 50 mmHg if measured internally, contractions palpated as “moderate” or greater would likely peak at 50 mmHg or greater if measured internally.

Resting tone: Average resting tone during labor is 10 mmHg; if using palpation, should palate as “soft”, i.e., easily indented no palpable resistance.

Relaxation time: Relaxation time is commonly 60 seconds or more in the first stage, and 45 seconds or more in the second stage.

Montevideo Units (MVUs): One Montevideo unit is equal to exactly 1 mmHg, Montevideo Units are directly equal to pressure change in mmHg summed over a ten-minute window. It is calculated by internally measuring peak uterine pressure amplitude (in mmHg) using an intrauterine pressure catheter, subtracting the resting tone of the contraction, and adding up the numbers in a 10-minute period. Usually range from 100-250 MVUs in the first stage, may rise to 300-400 MVUs in the second stage. Contraction intensities of 40 mm Hg or more and MVUs of 80-120 are generally sufficient to initiate spontaneous labor.

Assessment of uterine activity: Includes the identification of contraction frequency, duration, strength or intensity, and resting tone.

Tachsystole: Contraction frequency of greater than 5 in 10 minutes, averaged over 30 minutes; applies to spontaneous or stimulated labor.

Inadequate relaxation time: Less than 60 seconds uterine relaxation between contractions during the first stage of labor; less than 45-50 seconds uterine relaxation between contractions in the second stage.

No pushing (NP): No pushing mode.

### DESCRIPTION OF THE INVENTION

The present invention provides a uterine activity monitoring and assessment system 10 and methods of use thereof. The system 10 of this invention is designed to develop and support best practices in obstetrics, with a goal of providing clinicians with key information to help evaluate and decide on management of uterine activity at a patient's bedside. In a preferred embodiment, the system 10 will support a clinician's judgment, allowing the clinician to confirm bedside assessments.

Those skilled in the art and following the teachings herein provided will appreciate that while the descriptions below include preferred configurations, such configurations are used for illustrative purposes only and may be modified, as appropriate, depending on need.

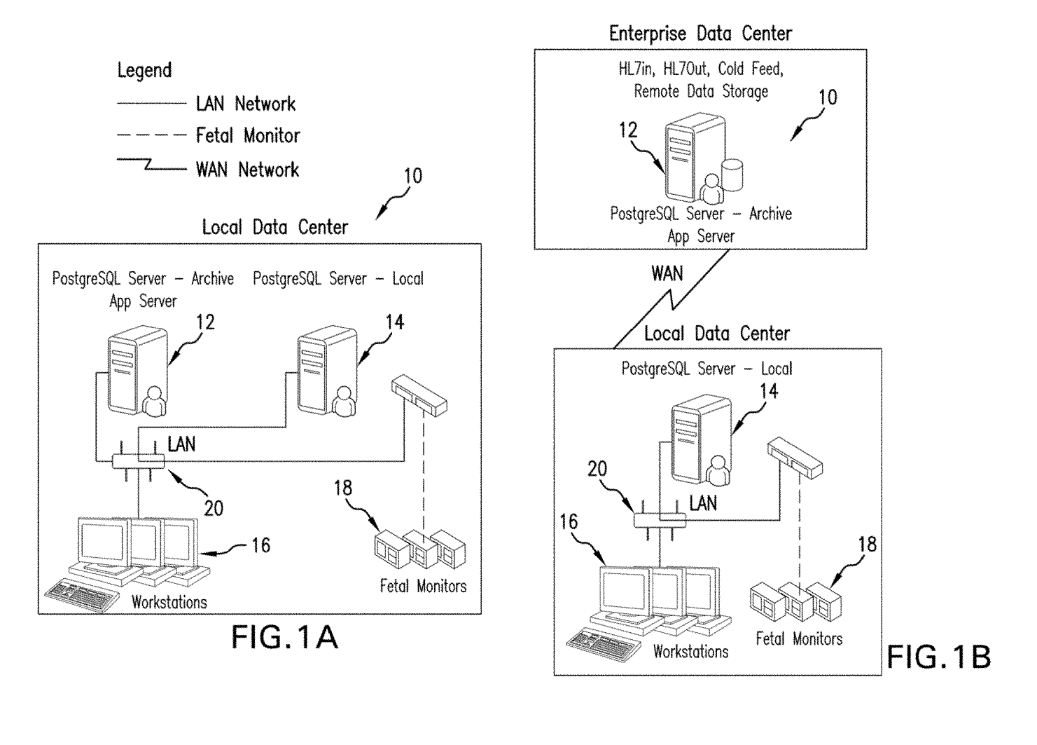

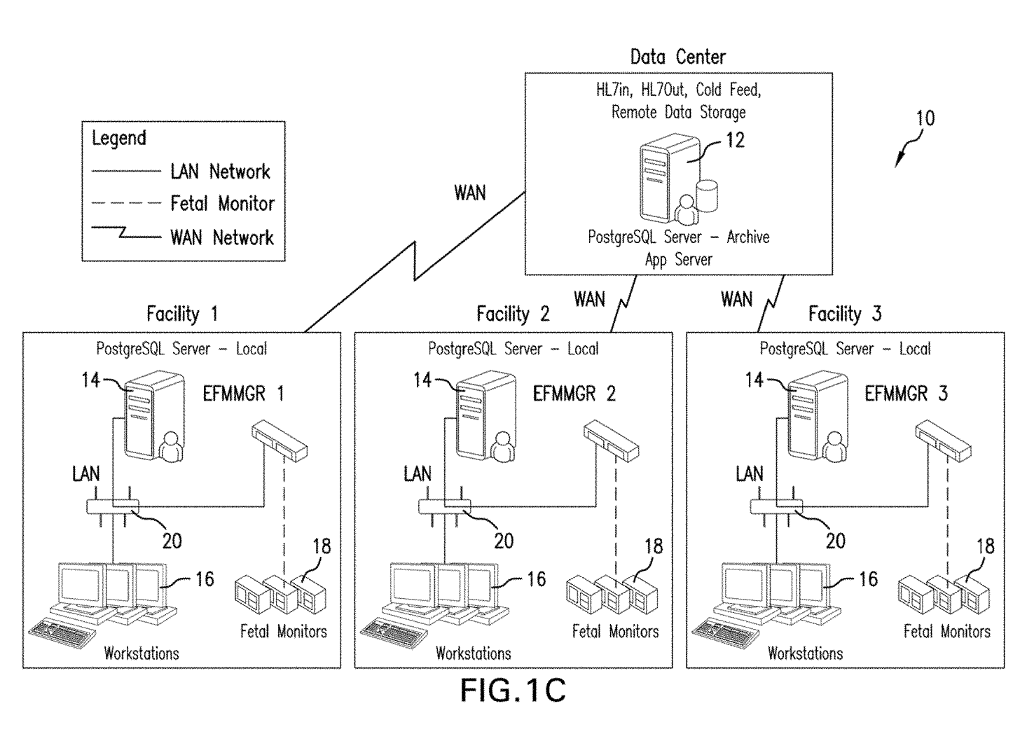

The uterine activity system 10 of this invention is designed to provide users, such as clinicians, with key information to help evaluate and decide on management of uterine activity of a pregnant patient, FIG. 1A shows a schematic representation of the uterine activity system 10 according to an embodiment of this invention. This embodiment comprises a system installed in a hospital's local data center that includes an archive app server 12, a local server 14, one or more workstations 16, and the hospital's fetal monitors 18. The archive/app server 12, the local server 14, the workstation 16, and the fetal monitor 18 are preferably connected with a local area network 20, however, other connections may be used. The workstation 16 preferably comprises one of a computer, a tablet, a smart phone or another device with a data processor, a monitor and an interface, such as a keyboard and/or a mouse. In an embodiment of this invention, the fetal monitor 18 may comprise a tocodynamometer external pressure sensor (TOGO) that connects to a patient. In another embodiment, the fetal monitor 18 may comprise an intrauterine pressure catheter (IUPC). FIG. 1B shows another schematic representation off the uterine activity system 110 according to another embodiment of this invention. This embodiment comprises an enterprise data center with the archive/app server 12 remote from a local data center and connected to the local data center with a wide area network. The local server 14, the workstation 16, and the fetal monitor 18 are preferably connected with a local area network 20. FIG. 1C shows another embodiment of the uterine activity system 10 of this invention. This embodiment is a multi-facility configuration with a remote data center connected to a plurality of facilities with a wide area network. Each of the facilities include a local server 14 connected to workstations 16 and fetal monitors 18 with a local area network.

In another embodiment, the uterine activity system 10 may comprise the workstation 16 connected to the fetal monitor 18. In this embodiment, the workstation includes an interface, a data processor and memory sufficient to operate the uterine activity system 10 of this invention. This embodiment may not include servers.

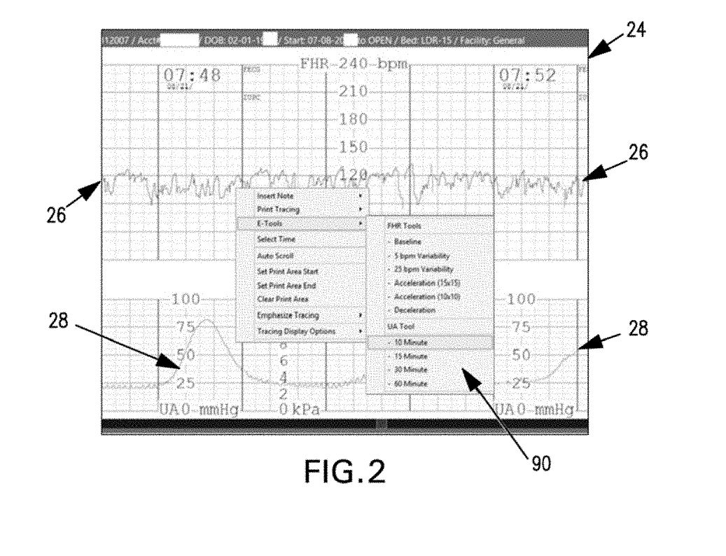

The uterine activity system 10 allows a clinician operating the workstation 16 to select and utilize a uterine contraction evaluation tool during a patient's intrapartum period. The clinician begins by logging into the system from the workstation 16. The clinician then selects a patient, and loads an associated trace. The system will preferably work with either an active trace or an archived trace. FIG. 2 shows an embodiment of a trace review screen 24 on the workstation 16 monitor. The trace review screen 24 in this embodiment includes a fetal heart rate tracing 26 and a uterine activity tracing 28. As shown in FIG. 2, an uterine activity tool 30 (UA tool) may be accessed by right clicking on the trace review screen 24 to bring up a drop-down menu 90 and selecting E-Tools then UA Tool. The UA tool 30 also allows the clinician to select a segment time period of 10 minutes, 15 minutes, 30 minutes, and 60 minutes. However, the system may be set up to select other periods. Selecting the time period launches the uterine activity tool 30.

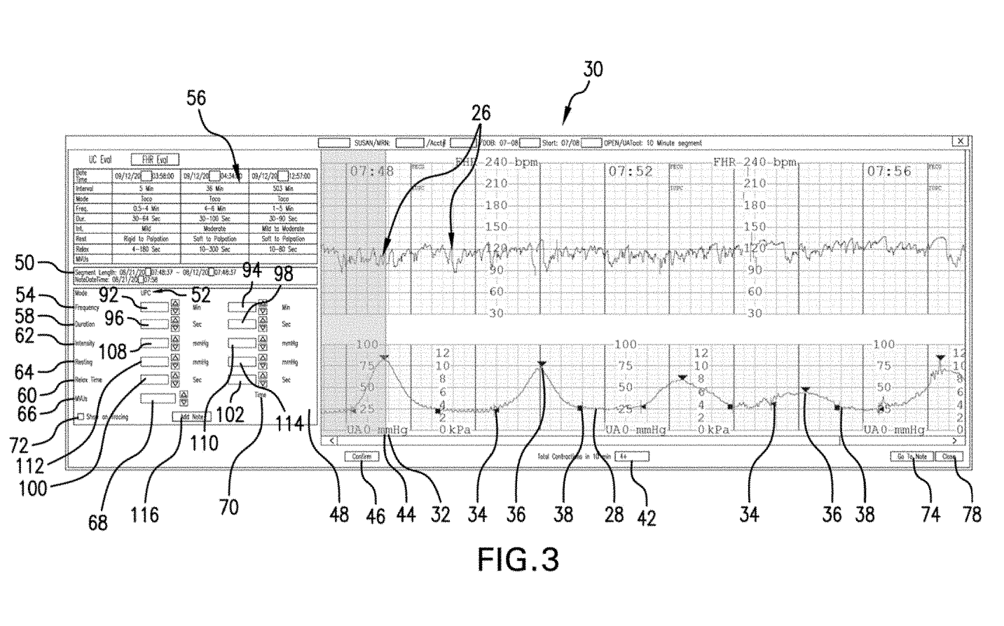

FIG. 3 shows a screenshot of the uterine activity tool 30 after a 10-minute period is selected from the trace review screen. In this embodiment, a segment range start line 32 appears at a beginning of a 10-minute segment on the uterine activity trace 28. The UA tool 30 preferably also places a segment range end line at the end of the trace, not shown in this figure. In this embodiment, the uterine activity tool 30 will display the selected ten-minute period and an additional accessible range. Please note in FIG. 3 the clinician lust scroll to see an entire length of the trace 28. In a preferred embodiment of this invention, an additional 2-minute period will be accessible beyond the end of the selected segment range. However, the additional accessible period may comprise any period of time.

In a preferred embodiment of this invention, the uterine activity tool 30 will detect if the fetal monitor 18 comprises one of a tocodynamometer external pressure sensor (TOCO) or an intrauterine pressure catheter (IUPC) that is connected to the patient and the fetal monitor or, in the case of a saved or archived trace, if the trace was created using the TOCO or the IUPC. The system will then display either TOCO or IUPC in the Mode field 52. If the system of this invention detects a trace that is a crossover of an IUPC and TOCO trace, then the system shall assume the segment range is all external and follow the external algorithm.

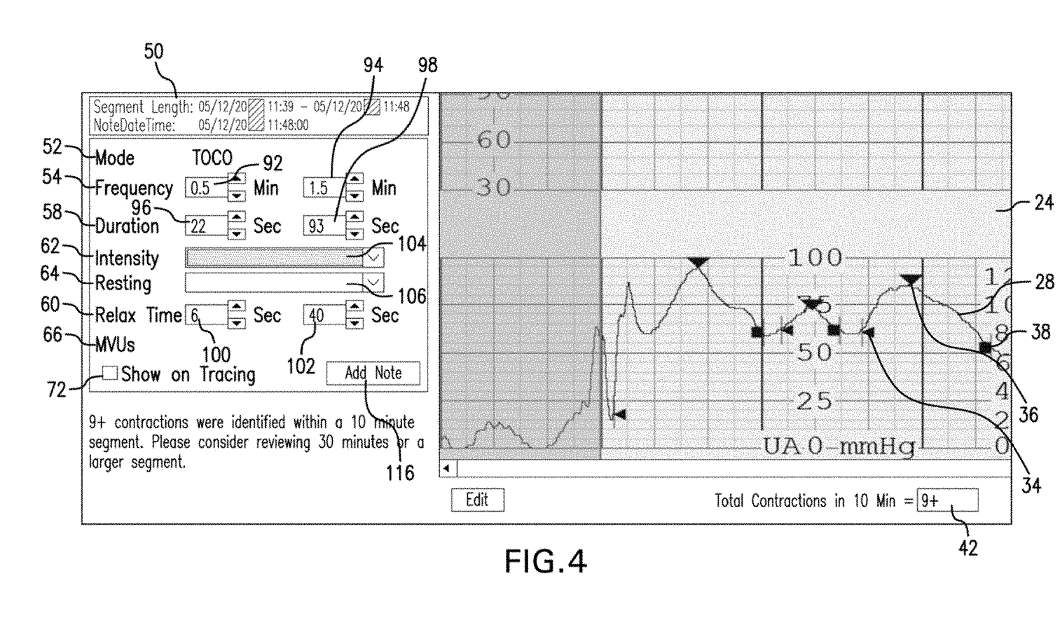

In a preferred embodiment of this invention, contraction identifying symbols, including a start 34, a peak 36 and an end 38, are calculated by the system and displayed on the uterine activity trace 28 within the segment range start line 32 and the segment range end line. In the embodiment shown in FIG. 3, the start 34 of a contraction is shown as a triangle pointing left with a line on the left side. The peak 36 of a contraction is shown as a triangle pointing down. The end 38 of a contraction is shown as square symbol with a line on the right side. The peak 36 is calculated as a midpoint between the start 34 of the contraction and the end 38 of the contraction. In a preferred embodiment, lengths of the uterine activity tracing 28 within the segment range that are not identified as a contraction will not display any contraction identifying symbols. The system 10 will also display any of the contraction identifying symbols 34, 36, 38 without detecting the other two symbols within the segment range start line 32 and the end line of the designated segment. If the system detects all three symbols 34, 36, 38 but only one or two of the symbols 34, 36, 38 are within the selected segment range, the uterine activity tool 30 may display the identified symbol or symbols however, this partial contraction in the segment will not be counted as part of a total count of contractions in the segment. In a preferred embodiment, the partial contraction will be indicated with a plus symbol. For example, FIG. 3 shows a contraction count window 42 showing “Total Contractions in 10 min=4+.” In an embodiment of this invention, when greater than 5 contractions have been detected in a 10-minute segment, the software may display a message to consider selecting a 30-minute or a larger segment. In another example, FIG. 4 shows the contraction count window 42 showing “9+”. FIG. 4 also shows a message to consider selecting a larger segment. Specifically, “9+ contractions were identified within a 10 minute segment. Please consider reviewing 30 minutes or a larger segment.”

In a preferred embodiment of this invention, after reviewing the contractions automatically marked by the UA tool 30, the clinician may adjust placement of the symbols denoting the start 34, the peak 36, and the end 38 of each contraction, based on clinical judgment. Upon confirmation by the clinician, the uterine activity tool 30 then displays vital information for assessment and documentation of uterine activity, including date, time, mode, interval, contraction frequency, contraction duration, contraction intensity, resting tone, relax time, calculated Montevideo Units (MVUs) if IUPC is in use, and number of contraction in the chosen time period. The clinician may then choose to accept the values provided by the UA tool 30, or edit these values prior to adding the values to the patient's chart.

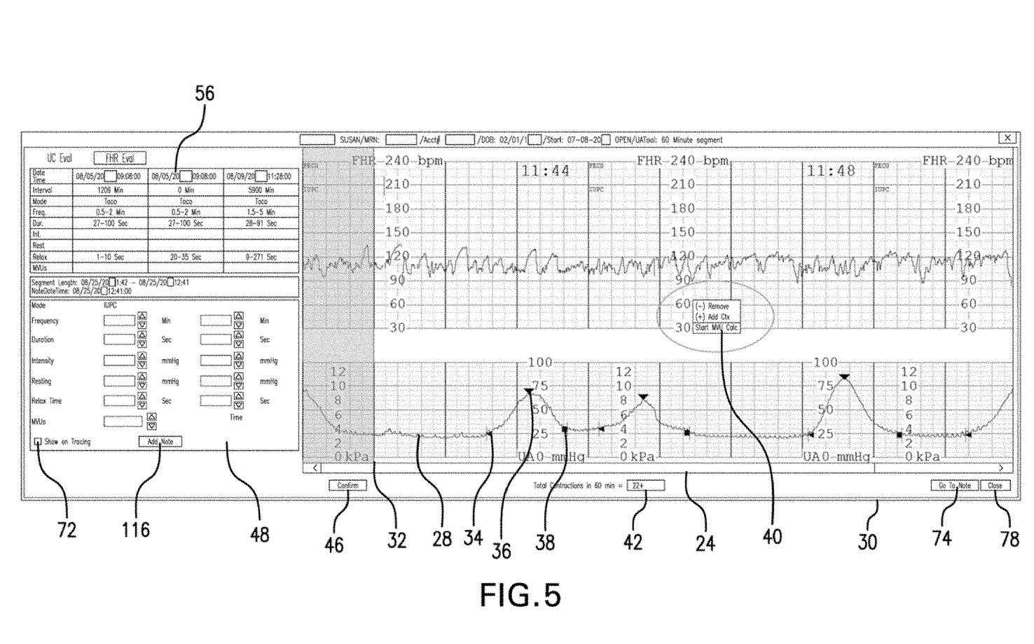

FIG. 5 shows the trace review screen 24 showing a context menu 40 for adjusting the symbols 34, 36, 38. The context menu 40 may include, for example but not limited to, selections for removing a contraction, repositioning the contraction symbols, adding a contraction, and starting MVUs calculation.

The method of removing one set of contraction identifiers starts by selecting one of the symbols 34, 36, 38 of the contraction that needs to be excluded and selecting the ‘(−) Remove’ option from the context menu 40. Selection of the symbol 34, 36, 38 may be accomplished by right-clicking on a mouse connected to the workstation 16. However, other methods and other types of interfaces may be used in connection with the workstation 16 to select the symbol 34, 36, 38. Upon selection, the context menu 40 automatically closes and all three contraction marking symbols for the selected contraction, are removed simultaneously. The contraction count window 42 at the bottom center of the UA Tool 30 web window is also preferably automatically updated based on the number of contractions removed. Note that the contraction markers are removed and the contraction is no longer included in calculations, but the uterine tracing is unchanged.

Repositioning one of the symbols 34, 36, 38 for the contractions can be accomplished by selecting the symbol with a left-click of the mouse and dragging the selected symbol to another position on the trace 28. Releasing the mouse button will set the symbol in a new position. Other methods and other types of interfaces may be used in connection with the workstation 30 to select the symbol and adjust the symbol as appropriate. In a preferred embodiment, a repositioned symbol will change color or otherwise indicate that the symbol has been manually changed to help a clinician differentiate repositioned symbols from those automatically detected by the system 10. The clinician user will be able to reposition multiple symbols however, preferably, the clinician may only reposition one symbol 34, 36, 38 at a time. Preferably, the symbol being moved will not be able to cross an x-coordinate of any other symbol on the trace 28. When the selected symbol is moved into an area that does not meet the specified contraction criteria, the selected symbol will preferably return to the original starting position.

In situations where the uterine activity tool 30 did not identify a contraction but the clinician visually identifies a contraction, the uterine activity tool 30 provides way for the clinician to add a contraction(s) prior to documenting. Adding the contraction starts by right-clicking on the uterine activity tracing 28 on the contraction that needs to be added. This step opens the context menu 40 and the clinician selects ‘(+) Add Ctx.’ The uterine activity tool 30 adds all three symbols 34, 36, 38 where the clinician clicked. The clinician must then reposition, as described above, the start symbol 34, the peak symbol 36, and the end symbol 38 as necessary to identify the contraction. In an alternative embodiment, the uterine activity tool 30 will identify and place the start symbol 34, the peak symbol 36, and the end symbol 38 at an appropriate position to identify the contraction. In a preferred embodiment, the uterine activity tool 30 will not allow the clinician to place a new contraction in the middle of another identified contraction. Preferably, when attempting to place a new contraction in the middle of another identified contraction, the ‘(+) Add Ctx’ option on the context menu 40 will be dimmed and unavailable for selection. In an embodiment of this invention, the clinician will not be able to add a new contraction outside of the segment. In a preferred embodiment, the manually added contractions will be identified with the symbols or a segment of the trace labeled with a new color or another identifier.

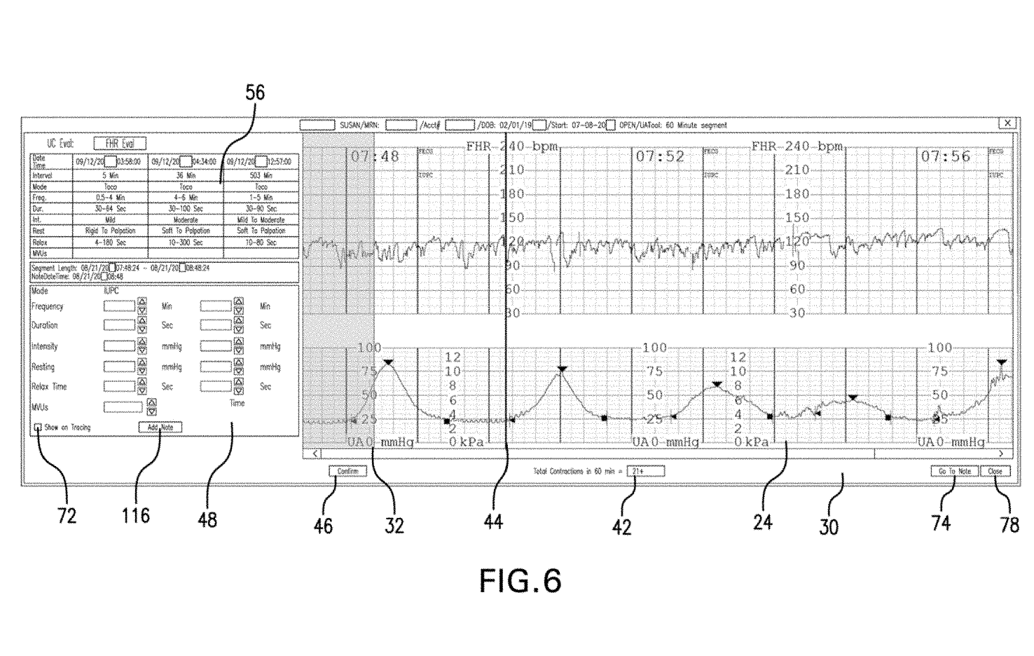

The uterine activity system of this invention preferably allows the clinician to automatically calculate Montevideo units (MVUs). MVUs are calculated by subtracting a baseline uterine pressure from a peak uterine pressure of each contraction in a 10-minute window of time and then taking the sum of these pressures. Two hundred Montevideo units or more is considered adequate for normal labor progression. The system of this invention calculates the MVUs when the intrauterine pressure catheter is used as the fetal monitor input 18. In a preferred embodiment, the clinician is unable to select ‘Start MVU Calc’ from the context menu 40 when the tocodynamometer external pressure sensor is used as the fetal monitor input 18. The clinician starts by accessing the patient on the workstation 16, selecting a segment for trace evaluation using the E-Tools context menu 90, and reviewing the patient's uterine activity tracing 28 on the trace review screen 24. The clinician may then select, preferably by right-clicking, a start point for calculating MVUs 44 on the uterine activity trace 28 and selecting ‘Start MVU Calc’ on the context menu 40. In a preferred embodiment, in order for MVUs to calculate, the clinician will need to select at least a 10-minute period. If the uterine activity tool 30 was selected for a 10-minute segment, the MVUs calculation start point 44 will be auto-selected from the beginning of the segment 32, if not selected, as shown in FIG. 3. If the uterine activity tool 30 was selected for a 15, 30, or 60 minute segment, the clinician may select the MVUs calculation start point 44. In a preferred embodiment, as shown in FIG. 6, the MVUs calculation start point 44 will be identified with a vertical line extending through the start point 44 and distinguished with a color or other identifier to distinguish the start point 44 from other elements on the trace review screen. In a preferred embodiment of this invention, if the clinician selects the start point 44 that is in the middle of a contraction, the system will automatically move the MVU start line 44 to the start of the selected contraction 34. When the clinician selects the MVU start point 44 for MVUs calculation inside a partial contraction near the segment range start line 32, the start point for MVUs calculation 44 will move to the end of the contraction 38. The clinician will be able to reset the 10-minute calculation window by right-clicking on the UA Tool web window trace display 24 and selecting ‘Start MVUs Calc’ 44, moving the existing line to the new position for the start of MVU calculation.

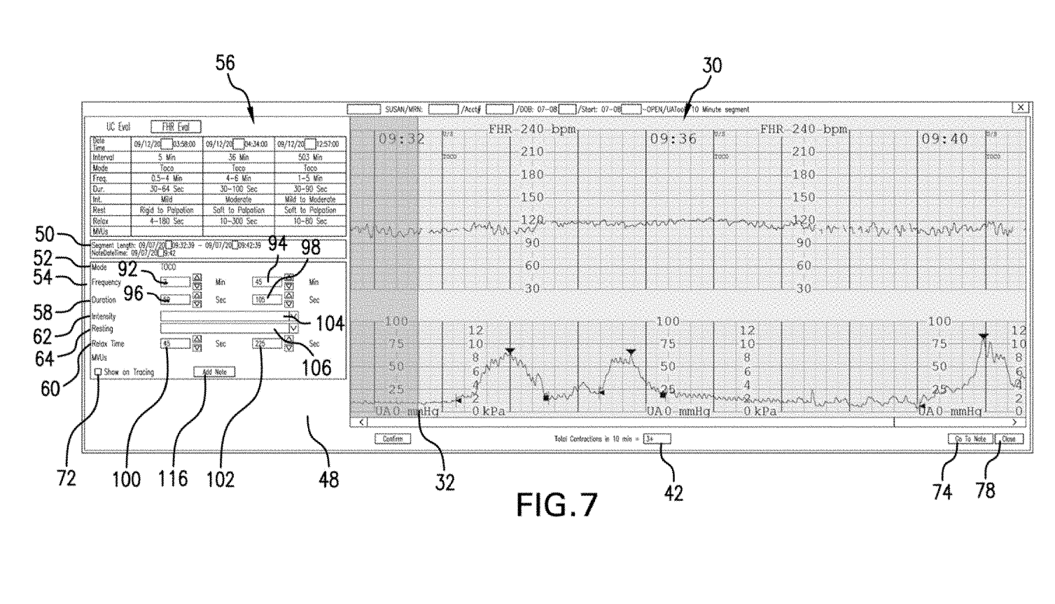

In a preferred embodiment of this invention, the clinician is able to confirm the identified contractions are accurate by selecting a Confirm button 46. During a confirmation process, the uterine activity system 10 will calculate a contraction data within the designated segment. In operation, the clinician accesses a patient's uterine activity tracing 28 on the workstation 16. The clinician assesses the uterine activity tracing 28 and modifies the contractions and/or tracing as necessary. When Assessment is completed, the clinician selects the confirm button 46 and a UC note entry section 48 becomes enabled, as shown in FIG. 7. Upon selection of the confirm button. 46, the uterine activity tool 30 processes contraction data within the selected segment to calculate frequency, duration, intensity, resting, relax, and MVUs (when the fetal monitor is an IUPC). In a preferred embodiment, the calculated data will auto populate into the appropriate UC Notes 48 entry fields. Preferably, partial contractions will not be included in the calculation. Contractions fully outside the segment range are not included in the calculations. If the section period is a 10-minute segment and the start point 44 for MVU calculations was set to a value other than the start of the segment, the uterine activity tool 30 will calculate the MVUs using contractions that are within the first minute following the segment end if the contraction is completed within 10 minutes from the selected start point 44 MVU calculations. In an embodiment of this invention, the confirm button 46 will be inactive when 1+ or fewer contraction are marked in the current segment range. In a preferred embodiment, a segment range display 50 is located above the UC notes entry section 48. As shown in the embodiment of FIG. 7, the segment range display preferably includes a segment length (start time to end time) and a notes entry time. In a preferred embodiment, at least some of the fields in the UC note entry section 48 are editable by the clinician. In a preferred embodiment, selection of the confirm button 46 also sends information to the patient's notes and flowsheets.

Preferably, a mode field 52 is automatically filled with TOCO when the fetal monitor 18 comprises a tocodynamometer external pressure sensor and the mode field 52 is automatically filled with IUPC when the fetal monitor 18 comprises an intrauterine pressure catheter (IUPC). The mode field 52 is preferably not editable by the clinician.

A Frequency Field Behavior field 54 is also shown in FIG. 7. In a preferred embodiment, the frequency field 54 is a required field that is automatically calculated by the uterine activity tool 30 when the confirm button 46 is selected. A first frequency text field 92 populates with a lowest contraction frequency automatically measured for all positively identified contractions within the segment range. A second frequency text field 94 populates with a highest contraction frequency measured. As shown in FIG. 7, the Frequency values calculated will be displayed in two spin box fields right of the “Frequency” label. The frequency will be calculated by averaging a time between start of a contraction to start of a next contraction within the segment range. The two frequency fields 92, 94 range from 0 to 300 minutes. In a preferred embodiment, the clinician may edit the frequency field 92, 94 values after the automatic calculation. In an embodiment, the frequency values may be increased and/or decreased using the spin box controls in increments of 0.5 minutes.

In a preferred embodiment, the uterine activity tool 30 calculates and populates a duration behavior field 58. The duration behavior field 58 preferably includes a first duration text field 96 showing a shortest contraction duration measured for all positively identified contractions within the segment range. The duration behavior field 58 preferably also includes a second duration text field 98 showing a longest contraction duration measured for all positively identified contractions within the segment range. Duration values in each of the fields preferably range from 10 to 480 seconds. In a preferred embodiment, the clinician may edit the duration fields after the automatic calculation. In an embodiment, the duration values may be increased and/or decreased in increments of 5 seconds using spin box controls.

In an embodiment of this invention, the uterine activity tool 30 also calculates and populates a relax time behavior field 60. A Relax Time behavior is calculated as time between the end point and the start point of each pair of positively identified contractions and displays an average for the selected segment. As shown in FIG. 7, a first relax time text field 100 populates with a lowest contraction relax time automatically measured for all positively identified contractions within the segment range. A second relax time text field 102 populates with a highest contraction relax time measured for all positively identified contractions within the segment range. In this embodiment, the relax time fields range from 0 to 999 seconds. In an embodiment f this invention, the clinician may increase or decrease the value in the relax time fields in increments of 5 seconds using a spin box control.

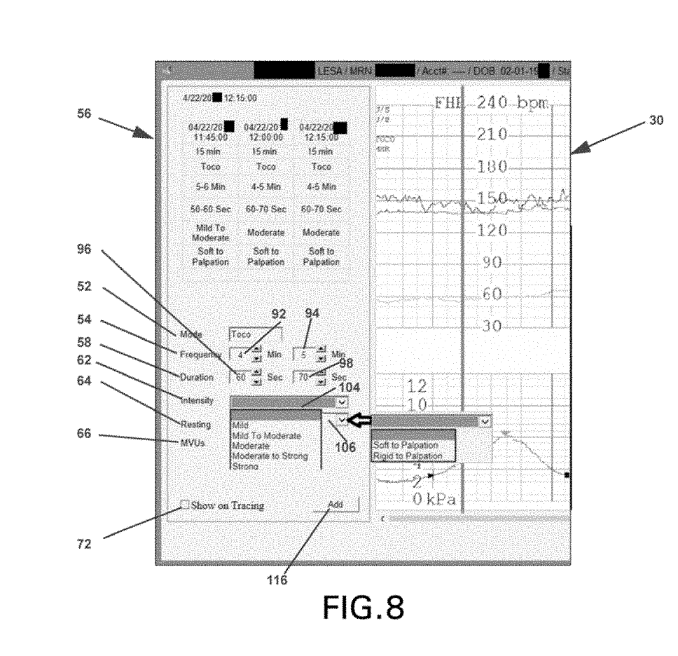

FIG. 8 shows a portion of the trace review screen 24 with the mode field 52 indicating Toco mode and showing the selection of an intensity field behavior 62. In a preferred embodiment, the intensity field behavior 62 is not a required field when in Toco mode. In this embodiment, the system of this invention will not auto-select a value for the intensity field behavior 62. Preferably, the intensity field behavior 62 includes an intensity field drop down box 104 for entering an intensity. Preferably, the Intensity field box is empty by default. For a value to be recorded in this field, the clinician selects a value from the drop down box. In a preferred embodiment, the drop down box allows for selection of one of mild, mild to moderate, moderate, moderate to strong, and strong. It should be noted that the intensity field behavior 62 may remain empty prior to adding data to notes.

FIG. 8 shows a portion of the trace review screen 24 with the mode field 52 indicating Toco mode and showing the selection of a resting field behavior 64. In a preferred embodiment, the resting field behavior 64 is not a required field for Toco mode. In this embodiment, the system of this invention will not auto-select a value for the resting field behavior 64. Preferably, the resting field behavior 64 includes a resting field behavior drop down box 106 for selecting an entry. Preferably, the resting field behavior drop down box 106 is empty by default. For a value to be recorded in this field, the clinician selects a value from the drop down box 106. In this embodiment, the clinician may select from the following options: soft to palpation or rigid to palpation.

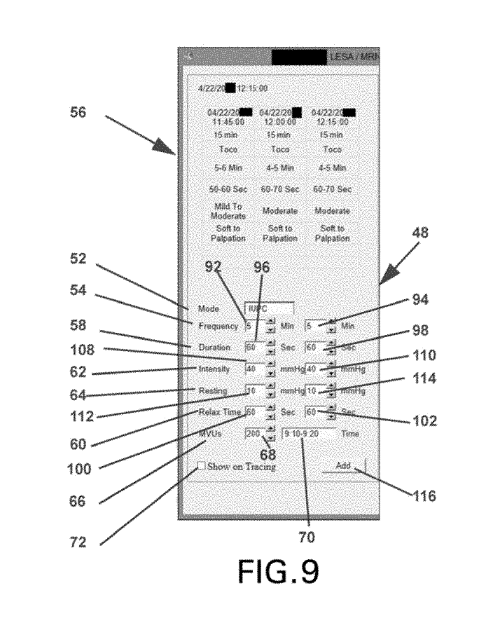

FIG. 9 shows a portion of the trace review screen 24 with the mode field 52 indicating IUPC mode. In a preferred embodiment, when in the IUPC mode, the intensity field behavior 62 field is a required field and the system of this invention automatically calculate the intensity field behavior and will automatically populate the intensity field 62. In the embodiment of FIG. 9, the intensity field behavior 62 includes a pair of intensity field behavior boxes. Preferably, a first intensity field behavior box 108 displays a lowest contraction intensity automatically measured for all positively identified contractions within the segment range. Preferably, a second intensity field behavior box 110 displays a highest contraction intensity automatically measured for all positively identified contractions within the segment range. In a preferred embodiment, the intensity field behavior is displayed in millimeter of mercury (mmHg). In an embodiment of this invention, the intensity field behavior 62 may be manually entered or adjusted. In this embodiment, the intensity field behavior 62 includes a spin box for entry or adjustment of the intensity field behavior 62. Alternatively, other types of input for entry or adjustment of the intensity field behavior may be used. In an embodiment of the invention, the spin box allows for selection of the intensity field behavior ranging from 0 to 100 mmHg and adjustable in increments of 1 mmHg. In one embodiment, a default value of the intensity field behavior is 40 mmHg.

FIG. 9 shows a portion of the trace review screen 24 with the mode field 52 indicating IUPC mode. In a preferred embodiment, when in the IUPC mode, the resting field behavior 64 is a required field and the system of this invention automatically calculate the resting field behavior and will automatically populate the field 64. In the embodiment of FIG. 9, the resting field behavior 64 includes a pair of resting field behavior boxes. Preferably, a first resting field behavior box 112 displays a lowest contraction resting automatically measured for all positively identified contractions within the segment range. Preferably, a second resting field behavior box 114 displays a highest contraction resting automatically measured for all positively identified contractions within the segment range. In a preferred embodiment, the resting field under Intensity will automatically display the resting range in millimeters of mercury (mmHg) upon selection of the document option. The values captured and calculated are all complete resting tones meaning the distance between the end of one contraction and the beginning of another. In an embodiment of this invention, the resting field behavior 64 includes a spin box for entry or adjustment of the resting field behavior. Alternatively, other types of input for entry or adjustment of the resting field behavior may be used. In an embodiment of the invention, the spin box allows for selection o f the resting field behavior ranging from 0 to 100 mmHg and adjustable in increments of 1 mmHg. In one embodiment, a default value of the resting field behavior is 10 mmHg.

When the system of this invention detects the IUPC is being used, a MVUs field behavior 6 box is enabled. In an embodiment, the MVUs field box 66 is not a required field. In a preferred embodiment, in order for the system to calculate MVUs, the clinician must select at least a ten-minute segment. Alternatively, the ten-minute segment will be auto-selected by the system. As shown in FIG. 9, the MVUs field behavior 66 includes two boxes: a MVU calculation 68 and a time field 70 when in IUPC mode. A time range for a selected MVU calc time segment will be shown in the time field 70. As discussed above, the MVU calculation 68 is calculated by measuring the peak intensity or amplitude (in mmHg) for each contraction occurring in a ten-minute window of time and adding the numbers together. Contraction amplitude is the difference between the resting tone and the peak of the contraction (in mmHg). For example, if there are three contractions in 10 minutes, peaking 70, 80, and 75 mmHg of intrauterine pressure, and the baseline uterine tone of 10 mmHg, this would be calculated as (70−10)+(80−10)+(75−10)=60+70+65=195 MVUs. In a preferred embodiment, the MVU calculation 68 includes a spin box for entry or adjustment of the MVU calculation 68. Alternatively, other types of input for entry or adjustment may be used. In an embodiment of the invention, the spin box allows for selection of the MVU calculation 68 ranging from 0 to 600 MVUs and adjustable in increments of 10 MVUs. If the MVU calculation is adjusted, the time field 70 is preferably cleared, indicating that the MVU calculation 68 has been adjusted. In one embodiment, a default value of the MVU calculations is 200. If a start MVU calculation time is not specified then MVUs will not be calculated and will show as blank (null) and no MVU Time will be displayed.

In a preferred embodiment of this invention, a UC note history section 56 is included in the uterine activity tool 30. In the embodiment shown in FIG. 7, the UC note history section 56 is positioned above the UC note entry section 48. The UC note history section 56 preferably includes a plurality of historical calculations each including a date time entry, an interval entry, a mode entry, a frequency entry, a duration entry, an intensity entry, a resting entry, a relax time entry, and an MVUs entry. In the embodiment shown in FIG. 7, the UC note history section 56 includes the last three sets of historical calculations. However, in alternative embodiments, the UC note history section 56 may include more or fewer sets of historical calculations. In the embodiment shown in FIG. 7, the UC note history section 56, a most recent historical calculation will populate a rightmost of the three columns, a second-most recent calculation will populate a center column and a third-most recent calculation will populate a leftmost column. In this embodiment, older calculations are not displayed.

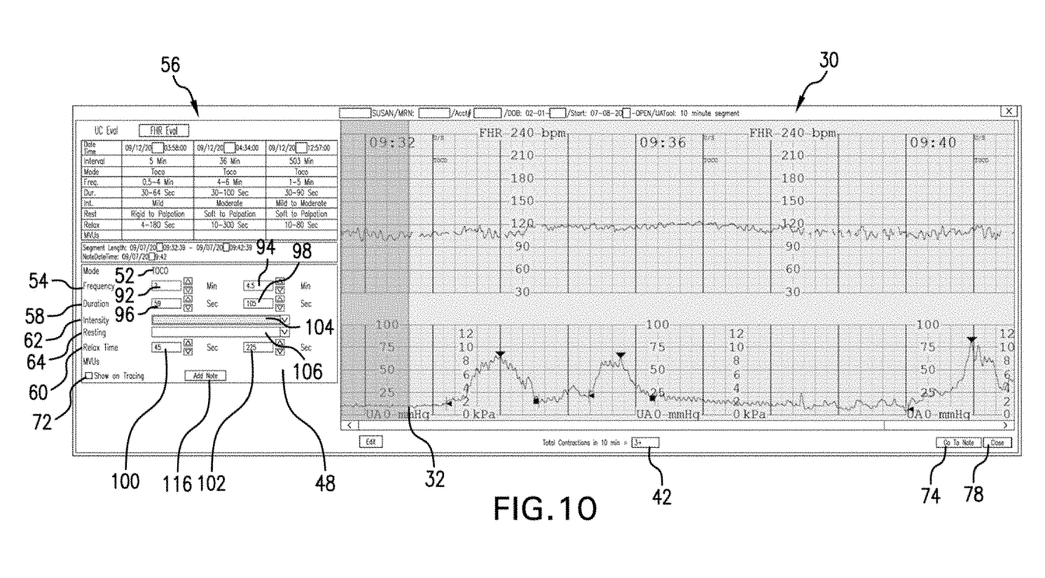

After data is collected, calculated and evaluated, the system 10 of this invention allows the clinician to save the data into patient's chart. Preferably, after saving, the data is available in notes and flowsheets. The clinician logs into the system 10, selects a patient, and displays the patient's associated uterine activity tracing 28. As previously described, the clinician uses the uterine activity tool 30 to select the UA trace segment range, evaluates the contractions, and selects the Confirm button 46 to confirm the contractions. Upon confirmation, the UC Notes Entry 48 will become active, an Add Notes button 116 becomes enabled, and a Go To Notes button 74 becomes enabled. As shown for example in FIG. 10, upon confirmation, the system of this invention will use the contraction data within the segment to calculate, for example, the frequency, the duration, the intensity, the resting, the relax, and the MVUs, if appropriate. In a preferred embodiment, two or more identified contractions within the segment will be required to calculate ranges. The UA Tool 30 calculated data will auto populate into the appropriate fields. Preferably, partial contractions will not be included in the calculations. Additionally, contractions fully outside the segment range are not included in the calculations. As shown in FIG. 10, in the top left of UC Notes Entry 48, the date and time of the end segment shall be displayed as the note time in notes. For example if the segment range starts at 12:00 pm and ends at 12:15 pm (15 minute segment), then the Note Time shall be: mm/dd/year 12:15:00.

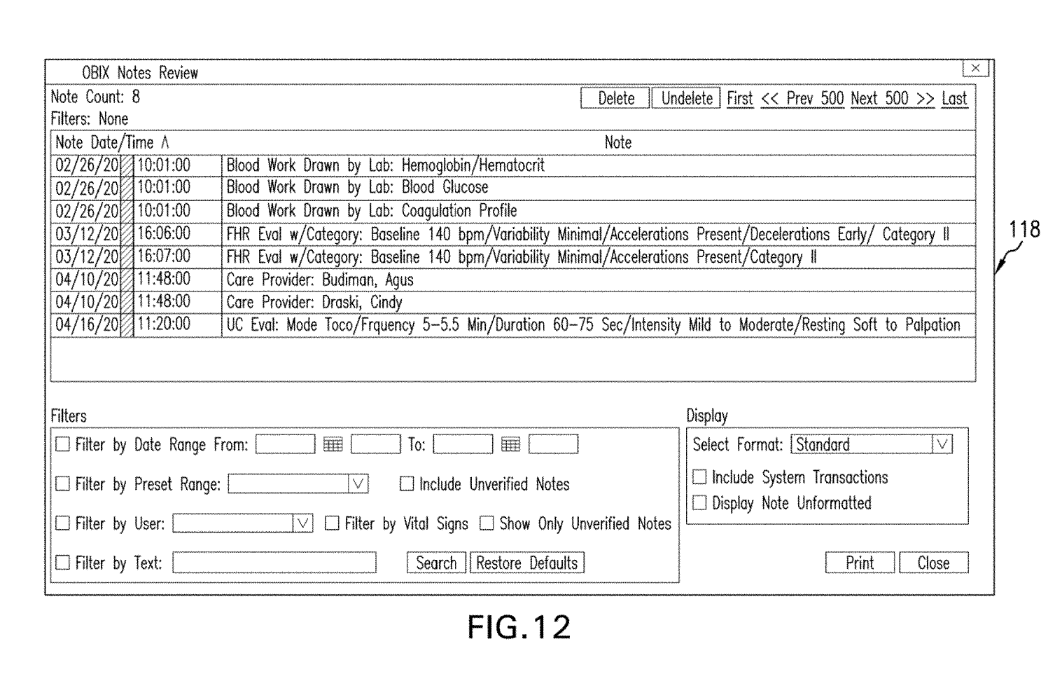

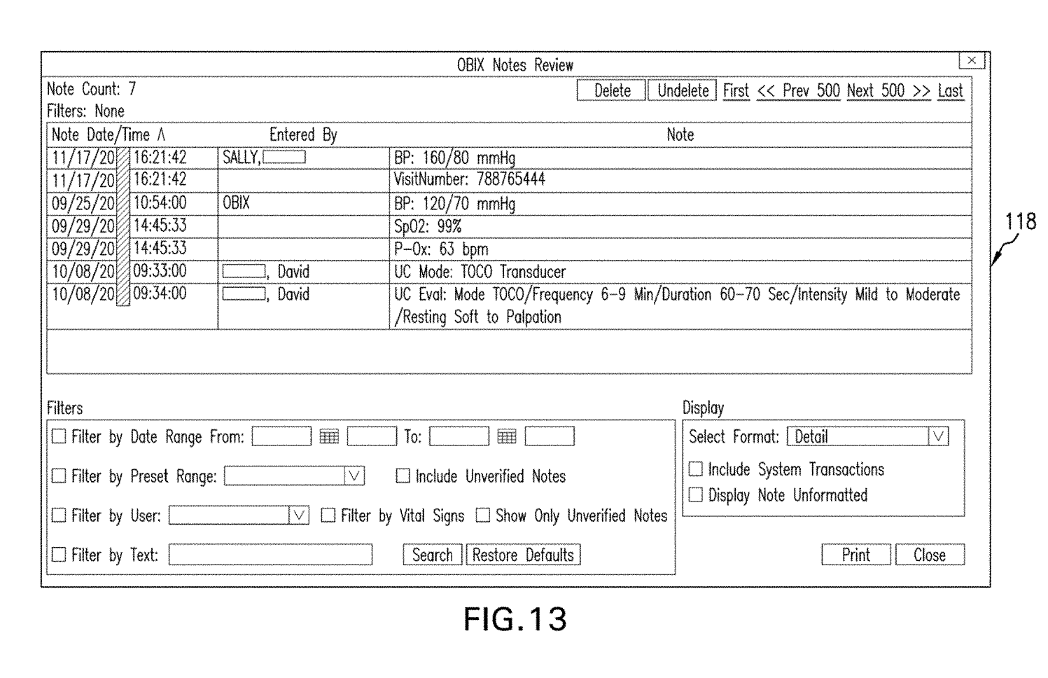

In the embodiment shown in FIG. 10, the UC notes entry section 48 further includes a Show on Tracing checkbox 72 having the same functionality as adding new contraction evaluation information from notes. Preferably, the Show on Tracing checkbox 72 is unchecked by default. The notes entry window 120 may also include the Show on Tracing checkbox 72 for information entered using the entry window 126. When selecting the Show on Tracing checkbox 72, an associated value will be captured and saved to the database of the system and will also be available for review on Notes Review 118, for example as shown in FIGS. 12 and 13.

When the UC Notes Entry 48 is first enabled, all the fields will be populated with the in formation just captured from the UA Tool 30. Under Toco mode, the intensity field behavior 62 and the resting field behavior 64 text boxes will not be auto-populated. The clinician will need to select values from the drop down boxes 104, 106. In a preferred embodiment, if the user removes a value leaving a required field empty, the add notes 116 button will become disabled, “greyed out”, until the required fields are populated. For the Toco mode, the required fields include frequency 54 and duration 58. For the IUPC mode, the required fields elude frequency 54, duration 58, intensity 62, and resting 64. When the required values are reentered, the add notes 116 button becomes enabled. Clicking the “add note” button 116 preferably causes the following actions to occur: UC Trending updates, values are cleared and Add button becomes disabled, the system sends all values to notes, and notes generated by UA Tool will be available to reports. Flow sheets will also be configured to display the uterine contraction values.

When adding information into the system, whether through entry into the UC notes entry 48 and/or the notes entry 120, the system of this invention shall keep track of the date, time, and user. The note time entered for the note will represent the end time and date of the selected segment range (i.e., in the 11:56-12:06 segment range, the time charted shall be 12:06 pm—late entry). The system will also keep track of the entry time when the user selects the Add option. In another embodiment, there will be no identification of the segment length in notes.

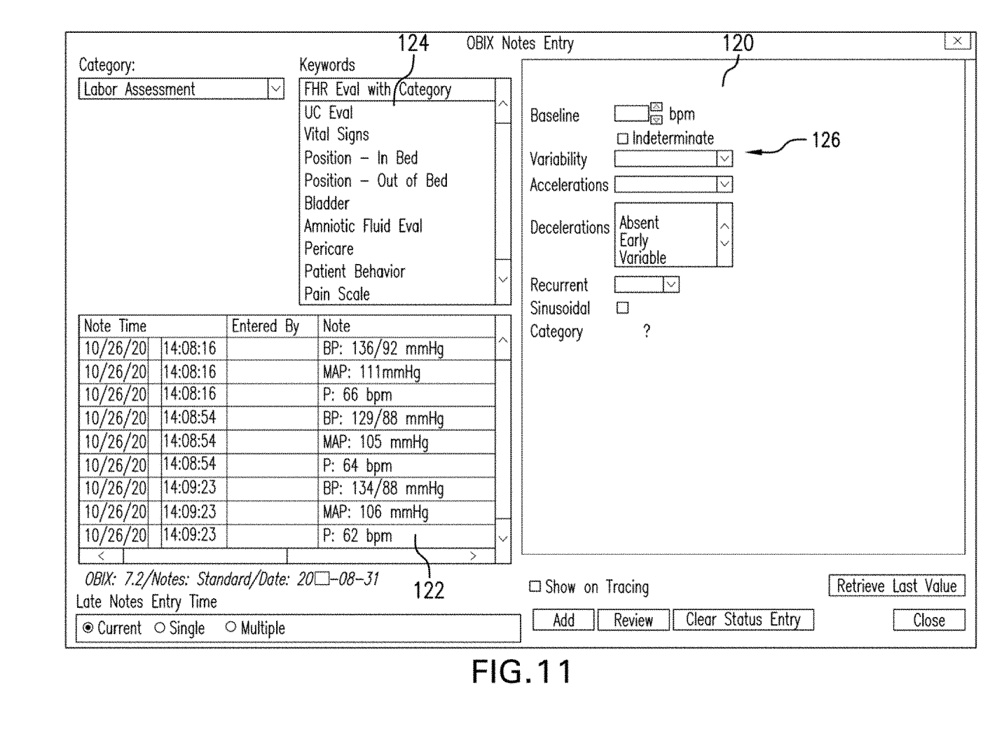

In a preferred embodiment, the clinician, after reviewing the uterine contraction evaluation information, is able to go directly to a notes module to continue charting information. As shown in FIG. 10, a Go To Note button 74 is located in the lower right hand corner of the uterine activity tool 30. In an embodiment of this invention, selecting the Go To Notes button 74 closes the uterine activity tool 30 and opens a notes entry window 120. FIG. 11 shows an embodiment of the notes entry window 76. In this embodiment, the notes entry window 120 displays the latest charted entries in a review window 122 and allows the clinician to select a keyword 124 and enter related information in an entry window 126. The keyword allows for entry of a variety of information related to pregnancy. Most important for this application is the UC eval keyword and the associated information entry. The notes web window is preferably opened in a “late charting” mode for the segment selected. The default time will be set to the end of the selected segment range. Preferably, the clinician will be able to go back to real time if needed. The clinician should also be able to change date and time if necessary. This will preferably be a global change that will also affect late charting from the trace display. When the clinician clicks on the trace display 24 to do late charting he/she will also have the ability to change to single late or real time charting. Preferably, when adding information into notes and flowsheets, the system will keep track of the date, time, and user. When Notes web window is closed, focus returns to the trace display window 24.

The clinician may also close the uterine activity tool 30 without saving any of the information by selecting a Close button 78 as shown in FIG. 10. Upon selecting the close button 78 on the UA Tool web window, the screen closes and, preferably, any pending changes will be discarded and the tool 30 will close without saving any data. In a preferred embodiment, the system returns to the trace display window 24, at the starting point of the previously selected segment range.

In an embodiment of this invention, the clinician may need the ability to retrieve a last value entered in the keyword 124 UC Eval. The clinician logs in and selects a retrieve last value button 80 and the last values stored to the database are displayed, whether saved from the UC Eval Confirmation Screen or saved after a manual entry in the OBIX Notes nodule 120. Category and keyword used will be the same as current Notes/UC Eval. This function occurs on all Notes/maternal/intrapartum or intrapartum multiples/UC Eval keyword.

In a preferred embodiment of this invention, the clinician is able to view information that was charted using the add button from the UC Evaluation confirmation screen. In the embodiment shown in FIG. 11, the clinician selects a review button 82. Selecting the review button 82 opens an open notes review screen 84, as shown in FIG. 12.

In an embodiment of this invention, a date and time displayed will be an end of the segment range stored during the UC Notes Entry 120 process. For example, if the segment range at time of confirmation was 12:56-1:06 pm (Entry Time) then the time displayed on this row will be 13:06 (Note Time) as military time applies. In a preferred embodiment, the clinician logged into the system at the time of UC Notes Entry will be the clinician displayed in notes review. In a preferred embodiment, the notes review/complete (including type) will remain the same as an evaluation added from the Notes module 120. When the user clicks “Add” on the tool to chart, the type, status, and trace column codes will remain the same as if the user utilized Notes to chart. In a embodiment of this invention shown in FIG. 13, the row of data will display fields in the following order and format: mm/dd/yyyy hour:min:sec; Clinician name; UC Eval: Mode/Frequency xx/Duration xx/Intensity xx/Resting xx/Relax time (if displayed)/MVUs xx.

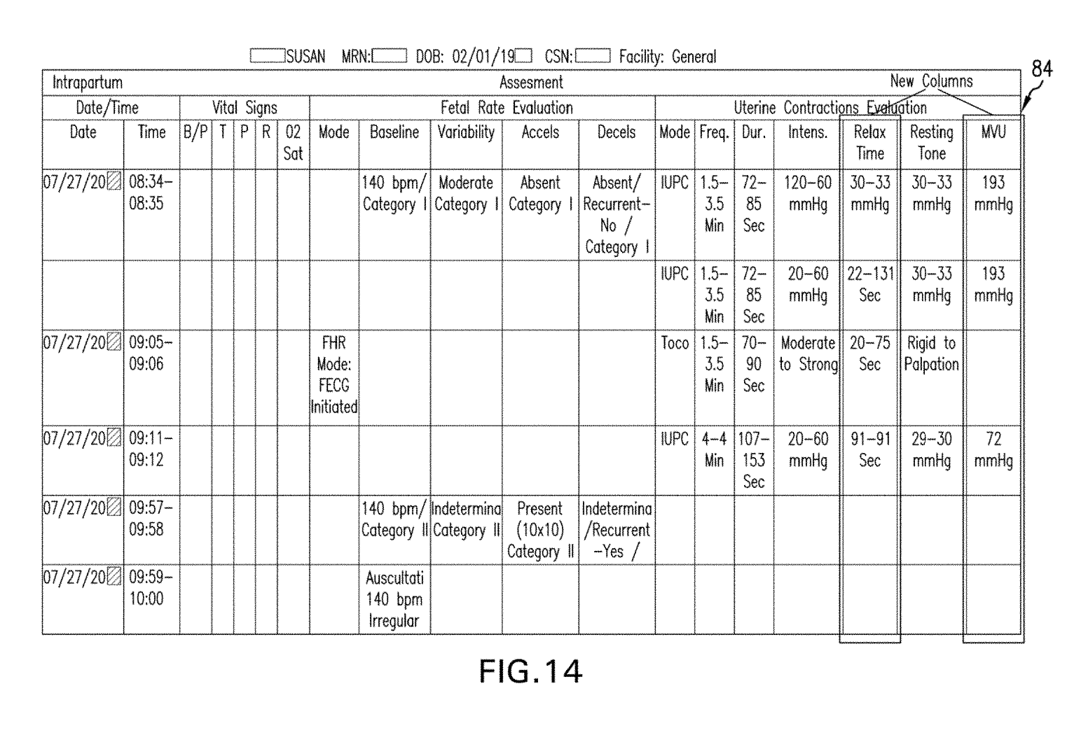

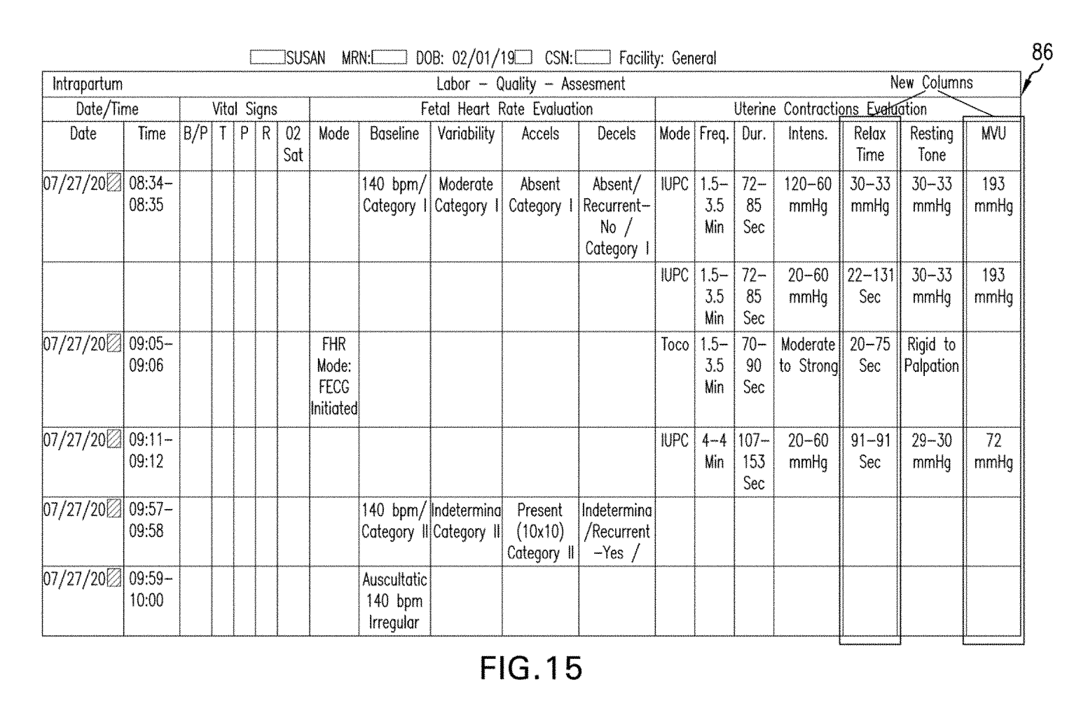

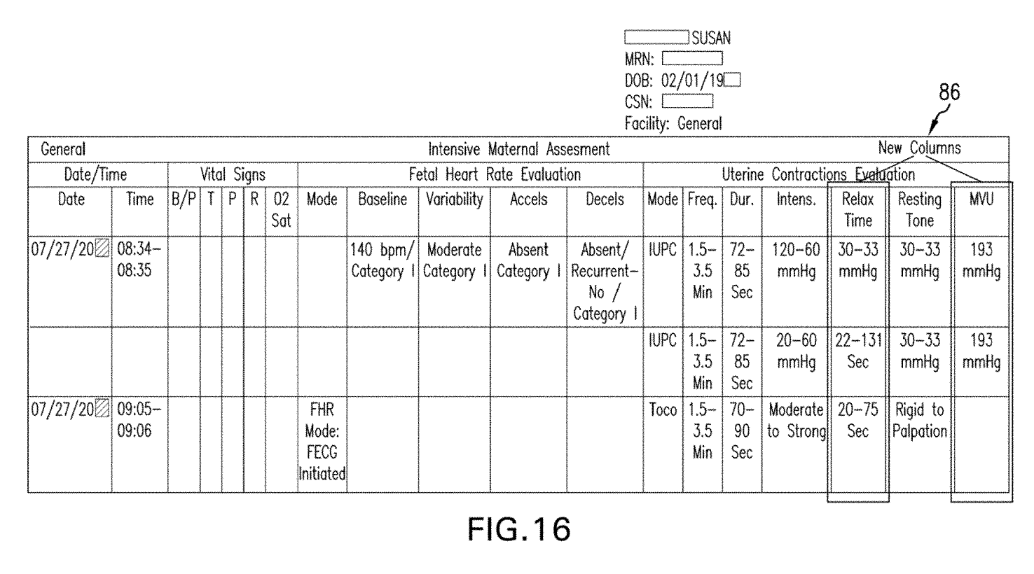

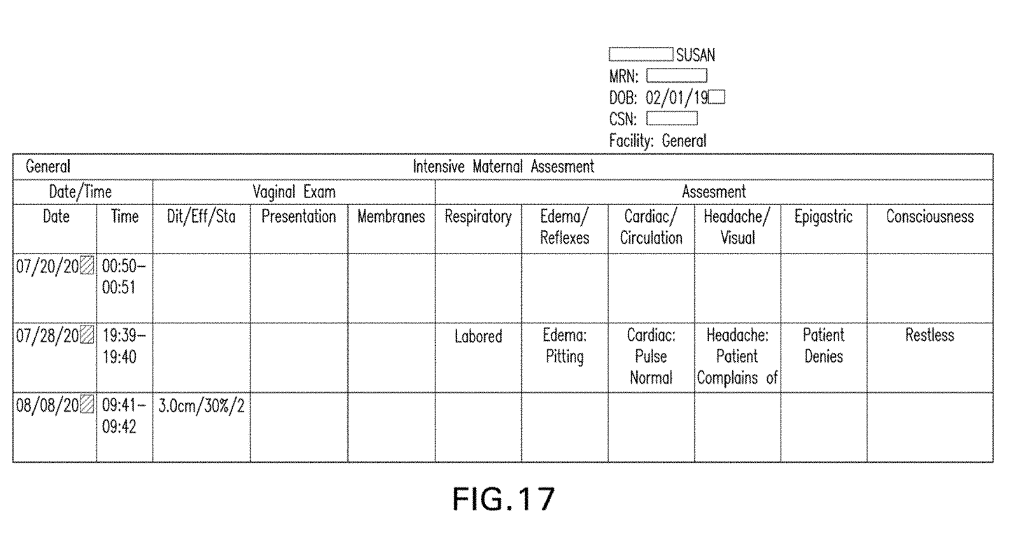

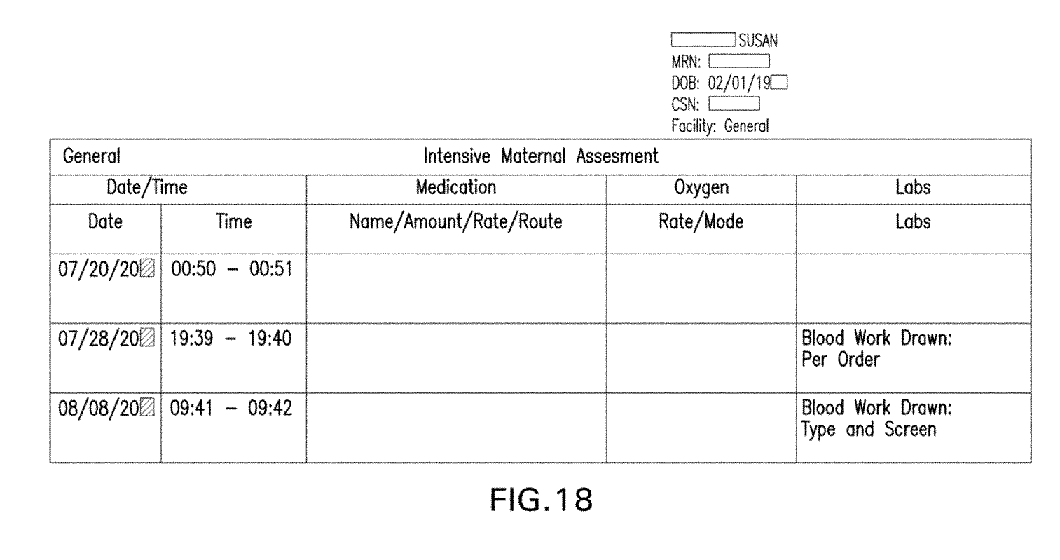

In a preferred embodiment, the system of this invention will allow a clinician to run a labor assessment and labor quality assessment flowsheet report 84 that includes all the information that was documented using the UA tool 30. The clinician will log in and access the UA tool 30. The clinician will then access flowsheets 84 by selecting Flowsheets>Intrapartum>Labor Assessment Uterine Contractions Evaluation section. FIG. 14 shows an embodiment of the flowsheet 84. When utilizing the UA tool 30, the left side of the chart of the flowsheet 84 shows a Uterine Contraction Evaluation section 128. In an embodiment of this invention, the Uterine Contraction Evaluation section 128 includes a plurality of columns including columns for mode, frequency, duration, intensity, relax time, resting tone, and MVU. In a preferred embodiment, all of the values stored from the UC Evaluation Confirmation section 128 will display in a single w in the flowsheet 84. Alternatively, as shown in FIG. 15, the clinician may choose to access another flowsheet 86 by selecting Flowsheets>Intrapartum>Labor Quality Assessment. In another embodiment, as shown in FIGS. 16-18, the clinician may choose to access another flowsheet 88 by selecting Flowsheets>Intrapartum>Intensive Maternal Assessment. This flowsheet 88 adds sections for Fetal Heart Rate Evaluation, Uterine Contractions Evaluation (including new fields as identified for Labor Assessment), Vaginal Exams and Oxygen.

The invention illustratively disclosed herein suitably may be practiced in the absence of any element, part, step, component, or ingredient, which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A system for monitoring, analyzing and documenting uterine activity, the system including:
at least one sensor positionable on or about a patient to measure maternal uterine contraction information including a uterine activity tracing;
an interface for at least one of entering or adjusting the maternal uterine contraction information and the uterine activity tracing;
a data processor in combination with the at least one sensor and the interface to receive and process the maternal uterine contraction information and the uterine activity tracing to identity one or more contractions in the maternal uterine contraction information; and
a display in combination with the data processor, wherein the display visually communicates to a clinician the maternal uterine contraction information and the uterine activity tracing including a start, a peak, and an end of each or the one or more contractions.

2. The system of claim 1, wherein the sensor comprises a tocodynamometer external pressure sensor.

3. The system of claim 1, wherein the sensor comprises an intrauterine pressure catheter.

4. The system of claim 1 wherein the start, the peak and the end of each of the one or contractions may be adjusted by the clinician as clinically appropriate.

5. The system of claim 1, wherein a clinician identifies a new contraction on the uterine activity tracing and adds labels to the new contraction on the uterine activity tracing.

6. The system of claim 1, wherein the clinician selects a segment period for assessment and documentation of the maternal uterine contraction information and the uterine activity tracing.

7. The system of claim 6, wherein the data processor calculates pregnancy information for assessment and documentation of the maternal uterine contraction information upon selection of a confirmation button by the clinician.

8. The system of claim 7, wherein the pregnancy information includes one or more of a date, a time, a mode, a contraction frequency, a contraction duration, a contraction intensity, a resting tone, a relax time, a calculated Montevideo Units (MVUs), and a number of contractions in a chosen time period.

9. The system of claim 8, wherein the information calculated is provided to one or more of a notes review or a flowsheet for review.

10. A method of monitoring, analyzing and documenting uterine activity, the method comprising the steps of:
acquiring maternal uterine contraction information including a uterine activity tracing;
displaying the material uterine contraction information including the uterine activity tracing at a workstation;
processing the maternal uterine contraction information and the uterine activity tracing to identify one or more contractions in the maternal uterine contraction information; and
calculating pregnancy information including one or more of a date, a time, a mode, a contraction frequency, a contraction duration, a contraction intensity, a resting tone, a relax time, a calculated Montevideo Units (MVUs), and a number of contractions in a chosen time period.

11. The method of claim 10 further including a step of entering or adjusting the maternal uterine contraction information and the uterine activity tracing.

12. The method of claim 10, wherein the step of acquiring maternal uterine contraction information utilizes a tocodynamometer external pressure sensor.

13. The method of claim 10, wherein the step of acquiring maternal uterine contraction information utilizes an intrauterine pressure catheter.

14. The method of claim 10 including a step of selecting a segment period for assessment and documentation of the maternal uterine contraction information and the uterine activity tracing.

15. The method of claim 10 further including a step of saving the calculated pregnancy information to one or more of a notes review or a flowsheet for review.

16. The method of claim 10 further including a step of idea identifying a new contraction on the uterine activity tracing and labeling the new contraction with a start, peak, and end point of the new contraction on the uterine activity tracing.

17. A method of monitoring, analyzing and documenting uterine activity, the method comprising the steps of:
acquiring with a sensor maternal uterine contraction information including a uterine activity tracing;
selecting a segment time range for analysis of the maternal uterine contraction information and the uterine activity tracing, wherein the segment time range comprises one of a 10-minute period, a 15-minute period, a 30-minute period, and a 60-minute period;
displaying the material uterine contraction information including the uterine activity tracing at a workstation;
processing the maternal uterine contraction information and the uterine activity tracing to identify one or more contractions in the maternal uterine contraction information;
entering or adjusting the maternal uterine contraction information and the uterine activity tracing;
confirming the maternal uterine contraction information;
calculating a number of contractions in a chosen segment time range;
calculating pregnancy information including one or more of a date, a time, a mode, a contraction frequency, a contraction duration, a contraction intensity, a resting tone, a relax time, and a calculated Montevideo Units (MVUs) for the chosen segment time range.

18. The method of claim 17, wherein the sensor comprises a tocodynamometer external pressure sensor.

19. The method of claim 17, wherein the sensor comprises an intrauterine pressure catheter.

20. The method of claim 17 further including a step of saving the calculated pregnancy information to one or more of a notes review or a flowsheet for review.
